(19) United States
(12) Patent Application Publication
(10) Pub. No.: US 2025/0228807 A1
(43) Pub. Date: Jul. 17, 2025

(54) TRANEXAMIC CLASS ARGININE AND HISTIDINE MIMICS AS THERAPEUTIC AGENTS

(71) Applicant: Tranexamic Technologies, LLC, Dallas, TX (US)

(72) Inventors: W. Paul Stewart, Dallas, TX (US); Frank Murdock, Dallas, TX (US)

(73) Assignee: Tranexamic Technologies, LLC, Dallas, TX (US)

(21) Appl. No.: 18/703,235

(22) PCT Filed: Mar. 27, 2023
(86) PCT No.: PCT/US2023/016391

§ 371 Date: Apr. 19, 2024

Related U.S. Application Data
(60) Provisional application No. 63/324,115, filed on Mar. 27, 2022.

(51) Int. Cl. A61K 31/196; A61K 31/215; A61P 35/00

(52) U.S. Cl. CPC A61K 31/196; A61K 31/215; A61P 35/00

(57) ABSTRACT

In various embodiments, the present disclosure pertains to compositions and methods for prevention or treatment of cancer, prevention, treatment or inhibition of viral or microbial infection or replication, or prevention, treatment or inhibition of other disease. In some embodiments, the composition includes at least one of an arginine mimic (TA-R) or a histidine mimic (TA-H) or the administration thereof. In some embodiments, the at least one TA-R or TA-H is a TA-R that can include, without limitation, trans-4-guanidinomethylcyclohexanecarboxylic acid (TA-R1), amino((((1r,4r)-4-(methoxycarbonyl)cyclohexyl)methyl)amino)methaniminium chloride (TA-R1-OMe), amino(((1r,4r)-4-carboxycyclohexyl)amino)methaniminium chloride (TA-R2), cis-amino(((3-carboxycyclohexyl)methyl)amino)methaniminium chloride (TA-R3), cis-amino(((3(methoxycarbonyl)cyclohexyl)methyl)amino)methaniminium chloride (TA-R3-OMe), and cis-3-(guanidinomethyl)cyclopentane-1-carboxylic acid (TA-R4), compounds that have a cycloalkane having two or more functional groups off the cycloalkane, derivates thereof, salts thereof, and combinations thereof.

### CROSS-REFERENCE TO RELATED APPLICATIONS

This patent application claims priority from, and incorporates by reference the entire disclosure of, U.S. Provisional Application 63/324,115 filed on Mar. 27, 2022.

### TECHNICAL FIELD

The present disclosure relates generally to therapeutic agents and more particularly, but not by way of limitation, to tranexamic class arginine and histidine mimics as therapeutic agents.

### BACKGROUND

This section provides background information to facilitate a better understanding of the various aspects of the disclosure. It should be understood that the statements in this section of this document are to be read in this light, and not as admissions of prior art.

Tranexamic acid (TA) is a clinically used agent that prevents plasmin formation by occupying the lysine (Lys) binding sites of plasminogen. TA is a synthetic analog of Lys created by interposing a cyclohexyl ring between the carboxyl and amino side chains of Lys. While historically used as an antifibrinolytic drug, it was recently shown that TA suppresses the viability of a broad set of human and murine cancer cell lines and inhibits the replication of multiple viruses and microbes.

### SUMMARY OF THE INVENTION

This summary is provided to introduce a selection of concepts that are further described below in the Detailed Description. This summary is not intended to identify key or essential features of the claimed subject matter, nor is it to be used as an aid in limiting the scope of the claimed subject matter.

In an embodiment, the present disclosure pertains to a composition for prevention or treatment of cancer. In general, the composition includes an arginine mimic (TA-R) that can include, without limitation, trans-4-guanidinomethylcyclohexanecarboxylic acid (TA-R1), amino((((1r,4r)-4-(methoxycarbonyl)cyclohexyl)methyl)amino)methaniminium chloride (TA-R1-OMe), amino(((1r,4r)-4-carboxycyclohexyl)amino)methaniminium chloride (TA-R2), cis-amino(((3-carboxycyclohexyl)methyl)amino)methaniminium chloride (TA-R3), cis-amino(((3 (methoxycarbonyl)cyclohexyl)methyl)amino)methaniminium chloride (TA-R3-OMe), and cis-3-(guanidinomethyl)cyclopentane-1-carboxylic acid (TA-R4), compounds that have a cycloalkane having two or more functional groups off the cycloalkane, derivates thereof, salts thereof, and combinations thereof.

In some embodiments, the arginine mimic has a concentration in a range of 0.5 to 20% (w/v). In some embodiments, the arginine mimic preserves a spatial relationship of a carboxyl group and a guanidinium group of an amino acid. In some embodiments, the arginine mimic inhibits viability of cells. In some embodiments, the cells include cancer cells. In some embodiments, the cancer cells are breast cancer cells. In some embodiments, the arginine mimic decreases expression of the MYC oncogene. In some embodiments, the arginine mimic reduces histone acetylation. In some embodiments, the arginine mimic inhibits p300 acetyltransferase activity. In some embodiments, the arginine mimic decreases programmed death-ligand 1 (PD-L1) expression in cancer cells thereby blocking T-cell-based immunity.

In a further embodiment, the present disclosure pertains to a method for prevention or treatment of cancer. In general, the method includes administering an arginine mimic (TA-R) to a subject. In some embodiments, the TA-R can include, without limitation, trans-4-guanidinomethylcyclohexanecarboxylic acid (TA-R1), amino((((1r,4r)-4-(methoxycarbonyl)cyclohexyl)methyl)amino)methaniminium chloride (TA-R1-OMe), amino(((1r,4r)-4-carboxycyclohexyl)amino)methaniminium chloride (TA-R2), cis-amino(((3-carboxycyclohexyl)methyl)amino)methaniminium chloride (TA-R3), cis-amino(((3 (methoxycarbonyl)cyclohexyl)methyl)amino)methaniminium chloride (TA-R3-OMe), and cis-3-(guanidinomethyl)cyclopentane-1-carboxylic acid (TA-R4), compounds that have a cycloalkane having two or more functional groups off the cycloalkane, derivates thereof, salts thereof, and combinations thereof.

In some embodiments, the arginine mimic has a concentration in a range of 0.5 to 20% (w/v). In some embodiments, the method further includes preserving, by the arginine mimic, a spatial relationship of a carboxyl group and a guanidinium group of an amino acid. In some embodiments, the method further includes inhibiting, by the arginine mimic, viability of cells. In some embodiments, the cells include cancer cells. In some embodiments, the cancer cells are breast cancer cells. In some embodiments, the method further includes decreasing, by the arginine mimic, expression of the MYC oncogene. In some embodiments, the method further includes reducing, by the arginine mimic, histone acetylation. In some embodiments, the method further includes inhibiting, by the arginine mimic, p300 acetyltransferase activity. In some embodiments, the method further includes decreasing, by the arginine mimic, programmed death-ligand 1 (PD-L1) expression in cancer cells and blocking T-cell-based immunity.

In an additional embodiment, the present disclosure pertains to a composition for prevention or treatment of cancer, prevention, treatment or inhibition of viral or microbial infection or replication, or prevention, treatment or inhibition of a disease. In general, the composition includes at least one of an arginine mimic (TA-R) or a histidine mimic (TA-H).

In some embodiments, the at least one of TA-R or TA-H is a TA-R that can include, without limitation, trans-4-guanidinomethylcyclohexanecarboxylic acid (TA-R1), amino((((1r,4r)-4-(methoxycarbonyl)cyclohexyl)methyl)amino)methaniminium chloride (TA-R1-OMe), amino(((1r,4r)-4-carboxycyclohexyl)amino)methaniminium chloride (TA-R2), cis-amino(((3-carboxycyclohexyl)methyl)amino)methaniminium chloride (TA-R3), cis-amino(((3 (methoxycarbonyl)cyclohexyl)methyl)amino)methaniminium chloride (TA-R3-OMe), and cis-3-(guanidinomethyl)cyclopentane-1-carboxylic acid (TA-R4), compounds that have a cycloalkane having two or more functional groups off the cycloalkane, derivates thereof, salts thereof, and combinations thereof. In some embodiments, the at least one of TA-R or TA-H has a concentration in a range of 0.5 to 20% (w/v). In some embodiments, the at least one of TA-R or TA-H preserves a spatial relationship of a carboxyl group and a guanidinium group of an amino acid. In some embodiments, the at least one of TA-R or TA-H treats or prevents the development or growth of cancer, a viral or microbial infection, or development of another disease. In some embodiments, the cancer is skin cancer. In some embodiments, the cancer is breast cancer. In some embodiments, the at least one of TA-R or TA-H decreases expression of the MYC oncogene. In some embodiments, the at least one of TA-R or TA-H reduces histone acetylation. In some embodiments, the at least one of TA-R or TA-H inhibits p300 acetyltransferase activity. In some embodiments, the at least one of TA-R or TA-H decreases programmed death-ligand 1 (PD-L1) expression in cancer cells thereby blocking T-cell-based immunity.

In a further embodiment, the present disclosure pertains to a method for prevention or treatment of cancer, prevention, treatment or inhibition of viral or microbial infection or replication, or prevention, treatment or inhibition of a disease. In general, the method includes administering at least one of an arginine mimic (TA-R) or a histidine mimic (TA-H) to a subject.

In some embodiments, the at least one of TA-R or TA-H is a TA-R that can include, without limitation, trans-4-guanidinomethylcyclohexanecarboxylic acid (TA-R1), amino((((1r,4r)-4-(methoxycarbonyl)cyclohexyl)methyl)amino)methaniminium chloride (TA-R1-OMe), amino(((1r,4r)-4-carboxycyclohexyl)amino)methaniminium chloride (TA-R2), cis-amino(((3-carboxycyclohexyl)methyl)amino)methaniminium chloride (TA-R3), cis-amino(((3 (methoxycarbonyl)cyclohexyl)methyl)amino)methaniminium chloride (TA-R3-OMe), and cis-3-(guanidinomethyl)cyclopentane-1-carboxylic acid (TA-R4), compounds that have a cycloalkane having two or more functional groups off the cycloalkane, derivates thereof, salts thereof, and combinations thereof. In some embodiments, the at least one of TA-R or TA-H has a concentration in a range of 0.5 to 20% (w/v). In some embodiments, the method further includes preserving, by the at least one of TA-R or TA-H, a spatial relationship of a carboxyl group and a guanidinium group of an amino acid. In some embodiments, the method further includes treating or preventing, by the at least one of TA-R or TA-H, the development or growth of cancer, a viral or microbial infection, or development of another disease. In some embodiments, the cancer is skin cancer. In some embodiments, the cancer is breast cancer. In some embodiments, the method further includes decreasing, by the at least one of TA-R or TA-H, expression of the MYC oncogene. In some embodiments, the method further includes reducing, by the at least one of TA-R or TA-H, histone acetylation. In some embodiments, the method further includes inhibiting, by the at least one of TA-R or TA-H, p300 acetyltransferase activity. In some embodiments, the method further includes decreasing, by the at least one of TA-R or TA-H, programmed death-ligand 1 (PD-L1) expression in cancer cells and blocking T-cell-based immunity.

### BRIEF DESCRIPTION OF THE DRAWINGS

A more complete understanding of the subject matter of the present disclosure may be obtained by reference to the following Detailed Description when taken in conjunction with the accompanying Drawings wherein:

FIG. 1 illustrates arginine mimics (TA-R) inspired by tranexamic acid (TA).

FIG. 2 illustrates TA-R compounds decrease breast cancer cell (MDA-MB-468) greater than tranexamic acid (TA); the cis-amino(((3-carboxycyclohexyl)methyl)amino)methaniminium chloride (TA-R3) curve is truncated due to insolubility.

### DETAILED DESCRIPTION

It is to be understood that the following disclosure provides many different embodiments, or examples, for implementing different features of various embodiments. Specific examples of components and arrangements are described below to simplify the disclosure. These are, of course, merely examples and are not intended to be limiting. The section headings used herein are for organizational purposes and are not to be construed as limiting the subject matter described.

As used herein, the term “tranexamic”—class, type of compound, or mimic—refers to compounds that have a cycloalkane having two or more functional groups off the cycloalkane. For example, in the case of tranexamic acid, a “lysine mimic”, the cycloalkane can be cyclohexane having two functional groups: (1) an aminomethyl group on the C₄ atom; and (2) a carboxylic acid group on the C₁ atom, thus having an amine group similar to that of lysine. In the case of an “arginine mimic”, the cycloalkane can be cyclohexane having two functional groups: (1) a guanidinomethyl group on the C₄ atom; and (2) a carboxylic acid group on the C₁ atom, thus having a guanidinium group similar to that of arginine. If should be noted that the aforementioned are examples only and various configurations are readily envisioned. For example, the cycloalkane can be cyclopentane, and the functional groups need not be on the C₄ and C₁ atoms. In addition, the functional groups are not limited to carboxyl groups, but can include various functional groups such as ester groups, for example. Further examples include, without limitation:
trans-4-guanidinomethylcyclohexanecarboxylic acid;
amino((((1r,4r)-4-(methoxycarbonyl)cyclohexyl)methyl)amino)methaniminium chloride;
amino(((1r,4r)-4-carboxycyclohexyl)amino)methaniminium chloride;
cis-amino(((3-carboxycyclohexyl)methyl)amino)methaniminium chloride;
cis-amino(((3 (methoxycarbonyl)cyclohexyl)methyl)amino)methaniminium chloride; and
cis-3-(guanidinomethyl)cyclopentane-1-carboxylic acid.

While historically used as an antifibrinolytic drug, it was recently shown that tranexamic acid (TA) suppresses the viability of a broad set of human and murine cancer cell lines, and inhibits the replication of multiple viruses and microbes. There are plasmin-dependent and plasmin-independent biochemical mechanisms responsible for this response, including inhibition of protein synthesis and suppression of lysine (Lys) acetylation. More particularly, it was also recently shown that the fundamental method of action (MOA) of TA is that it mimics (antagonizes) not just Lys but also arginine (Arg) and histidine (His), the three positively charged amino acids, which are essential for the electrostatic bonding required in multiple protein-protein interactions and other processes involved in cancer formation, the replication of viruses and microbes, and the development of other diseases and conditions.

In addition to blocking the Lys binding sites on plasminogen to inhibit the formation of plasmin, specific examples of this fundamental MOA that a have been demonstrated include, for example, inhibition of Lys acetylation (required for creation of proteins involved in viral replication and the development of cancer and other diseases) and occupation of cationic amino acid transporters (CATs) required by Lys, Arg, and His to be transported into cells, thereby depriving the cells of these amino acids and inhibiting the protein proliferation required for viral replication and cancer growth.

Based on the hypothesis that a synthetic “tranexamic” mimic of Arg or a synthetic “tranexamic” mimic His might be more effective than TA against certain cancers, viruses, or other diseases or conditions—whether because Arg or His plays a more important role in those cases or for other reasons—the present disclosure pertains to creation of various synthetic “tranexamic” mimics of Arg that were tested against a common breast cancer line. The present disclosure shows that all Arg mimics indeed were significantly more effective than TA in suppressing the viability of this type of cancer. As such, the present disclosure opens up the possibility of using synthetic “tranexamic” mimics of Arg and “tranexamic” mimics of His against viruses, microbes, cancers, and other diseases or conditions, when TA does not provide inhibition or sufficient inhibition, as well as using them in combination with TA or with other drugs to provide increased efficacy or safety.

Reported herein are the results of the development of several TA-R molecules including, for instance, biological testing. The following outlines various TA-R abbreviations used herein: trans-4-guanidinomethylcyclohexanecarboxylic acid (TA-R1); amino((((1r,4r)-4-(methoxycarbonyl)cyclohexyl)methyl)amino)methaniminium chloride (TA-R1-OMe); amino(((1r,4r)-4-carboxycyclohexyl)amino)methaniminium chloride (TA-R2); cis-amino(((3-carboxycyclohexyl)methyl)amino)methaniminium chloride (TA-R3); cis-amino(((3 (methoxycarbonyl)cyclohexyl)methyl)amino)methaniminium chloride (TA-R3-OMe); and cis-3-(guanidinomethyl)cyclopentane-1-carboxylic acid (TA-R4).

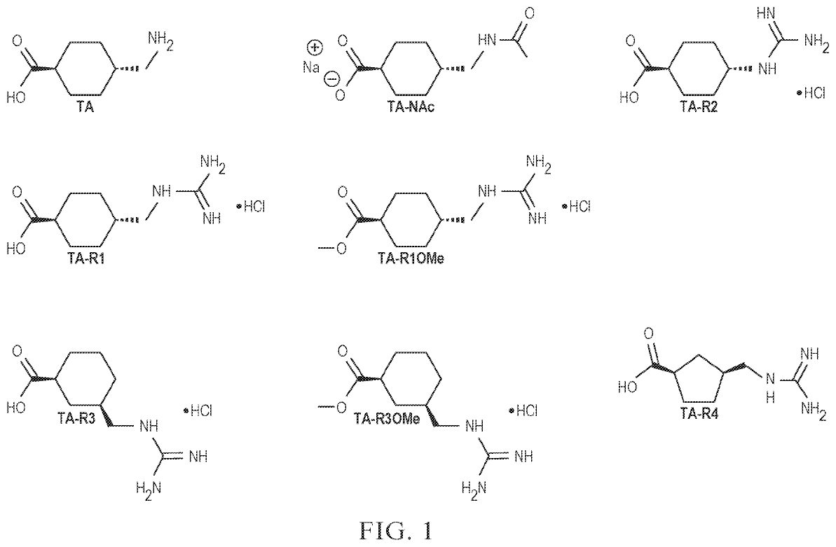

Arginine mimic design and compounds prepared. To a first approximation, TA mimics Lys by preserving the 1,6-connectivity between the carboxyl functional group and the amino group present on the Lys side chain. Known is that the trans stereochemistry of TA and the cyclohexyl ring are important for plasminogen binding, the former to preserve an extended conformation of the molecule and the latter to provide stabilizing van der Waals contacts within the Lys binding sites. By analogy, several Arg mimics were identified and biological evaluation that preserve the spatial relationship (e.g., 1,5-connectivity) of the amino acid's carboxyl and guanidinium groups (with the exception of TA-R1 and TA-R1OMe) were studied. These molecular targets are identified in FIG. 1 with the designation TAR and complemented by various important descriptors. Also shown are comparator compounds, TA and TA-NAc (N-acetylcysteine; NAc), evaluated in parallel.

### WORKING EXAMPLES

Reference will now be made to more specific embodiments of the present disclosure and data that provides support for such embodiments. However, it should be noted that the disclosure below is for illustrative purposes only and is not intended to limit the scope of the claimed subject matter in any way.

### Biological Testing

Effects of compounds on cell viability. MDA-MB 468 triple-negative breast cancer cells were plated at 6,250 cells per well in a 96-well plate and incubated overnight at 37° C. Cells were subsequently incubated for 72 h with increasing concentrations of each TA analog as indicated in FIG. 1 at 37° C. Cell viability was evaluated using the MTT (3-(4,5-dimethylthiazol-2-yl)-2,5-diphenyltetrazolium bromide) assay carried out according to manufacturer's instructions.

Inhibition of p300 acetyltransferase activity. p300 enzyme assays were carried out with TA, TA-NAc, and TA-R2 using commercially available recombinant p300, and recombinant, commercial Histone H3 and tritiated acetyl CoA as the substrates. P300 assays were performed in the presence of increasing concentrations of each compound and acetylation of p300 was evaluated by scintillation counting of Histone H3 adsorbed to 1 cm×1 cm nitrocellulose squares, after extensive washing of the nitrocellulose squares with 20% trichloroacetic acid.

### RESULTS AND DISCUSSION

Chemical synthesis and solubility. TA was purchased from commercial sources and TA-NAc was prepared straightforwardly through acetylation of TA based on standard practices. TA-NAc was isolated and tested as its sodium salt which displays better aqueous solubility.

TA-R1-R4 were accessible from their corresponding amino acids through treatment with methylisothiourea hemisulfate. TA-R1 (trans-4-guanidinomethylcyclohexanecarboxylic acid; GMCHA) was prepared in one step from TA using this reagent, but displayed poor aqueous solubility in its neutral form and as its salts. Esterification of TA-R1 to produce TA-R1OMe, a novel compound, improved the solubility for preparation of a 2% (w/v) solution in phosphate buffered saline (PBS) buffer (pH=7.4) and biological testing. TA-R2 was prepared in one step from commercially-available trans-4-aminocyclohexanecarboxylic acid and displayed good aqueous solubility.

The requisite amino acids for synthesis of TA-R3/R4 required multistep synthetic approaches. Briefly, the preparation of TA-R3, a novel compound, began from cis-1,3-cyclohexanedicarboxylic acid and proceeded through eight sequential synthetic steps to afford the product as its HCl salt. Protection/deprotection of the amino group was required to facilitate purification, as was a stepwise oxidation sequence from the alcohol to the carboxylic acid. The compound displayed poor aqueous solubility (˜0.1% in PBS buffer), but this was remedied by esterification to produce TA-R3OMe (to prepare a ca. 2% solution). The approach to TA-R4, a known compound, required one additional step to produce the starting dicarboxylic acid, in this case through oxidative cleavage of norbornene. TA-R4 demonstrated sufficient solubility in its neutral form (ca. 2% solution in PBS buffer). All the final compounds were fully characterized by ¹H nuclear magnetic resonance (NMR), ¹³C NMR, and high-resolution electrospray ionization mass spectrometry (HRMS-ESI).

### Biological Results

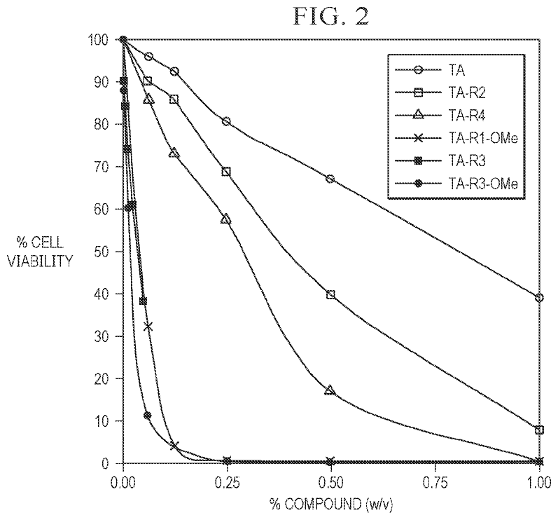

TA-R compounds suppress cancer cell viability in vitro. MTT cell viability assays showed that the tested TA-R compounds suppressed the viability of breast cancer (MDA-MB-468) cells (FIG. 2) to a greater extent than TA (or TA-NAc; data not shown).

Biochemical evaluation of treatment of breast cancer cells with TA-R2. Immunoblot analysis of treatment of MDA-MB-468 cells with TA-R2 versus TA and TA-NAc (NAc-TA) indicated that TA-R2 decreases expression of the MYC oncogene to a greater extent than TA or NAc-TA at the same concentrations. High (2%) TA-R2 decreases programmed death-ligand 1 (PD-L1), whereas the other two compounds do not. This is important because PD-L1 expression in cancer cells blocks T-cell-based immunity. So, TA-R2 would be predicted to increase anticancer immunity in immunocompetent systems. Histone acetyltransferase (HAT) enzyme assays were used to determine if TA-R2 inhibits p300 activity. While TA (2%) does not affect p300 acetylation of Histone 3 (H3) in vitro, TA-R2 decreases histone acetylation more strongly than the other two compounds. This fits with the hypothesis that TAs act in part by inhibiting acetyltransferases such as p300/CPB that acetylate histones. No acetylation is observed in controls upon omission of H3/p300 or acetyl coenzyme A (Acetyl CoA).

### CONCLUSIONS

All of the TA-R analogs tested exhibit a higher potency of cytotoxicity against the MDA-MB-468 triple-negative breast cancer cell line than TA with approximately 10-fold lower IC₅₀ values for TA-R1-OMe, TA-R3, and TA-R3-OMe. Difficulties with low solubility of some analogs was addressed by generating ester derivatives in some cases and in other cases by producing the compounds as salts (or both). One of the analogs tested, TA-R2, downregulated expression of the MYC oncogene and reduced histone acetylation to a greater extent than TA at the 2% (w/v) concentration, and unlike TA, TA-R2 downregulated MYC at 0.5%. Overall, these results indicate that replacing the amine group of TA or TA-inspired compounds with a guanidinium group may enhance anticancer potency and anticancer activity through biochemical mechanisms that overlap with those of TA.

Based on the results presented above, although TA has been shown to antagonize Lys, Arg, and His, it is clear that Arg mimics, such as TA-R molecules discussed above, are more effective than TA in certain circumstances. It is readily envisioned that additional circumstances exist. For example, Arg deprivation has been identified as a therapeutic method for additional types of cancer and for the virus that caused the coronavirus disease 2019 (COVID-19) pandemic (severe acute respiratory syndrome coronavirus 2; SARS-CoV-2). If TA-R is more effective than TA at antagonizing Arg, which is likely, then it will be more effective in these additional therapeutic areas.

Additionally, based on the data presented herein, it is further envisioned that use of the TA-R mimics discussed herein could provide a means to improve the efficacy of radiation treatment against certain tumor types, such as, for example glioblastoma, as Arg deprivation has been shown to improve the effectiveness of radiation treatment of those tumors. Furthermore, as Arg is also involved in aspects of the physiologic response to traumatic brain injury, the mimics disclosed herein could be utilized in treatment of brain injuries, including, for example, concussions.

Although various embodiments of the present disclosure have been illustrated in the accompanying Drawings and described in the foregoing Detailed Description, it will be understood that the present disclosure is not limited to the embodiments disclosed herein, but is capable of numerous rearrangements, modifications, and substitutions without departing from the spirit of the disclosure as set forth herein.

The term “substantially” is defined as largely but not necessarily wholly what is specified, as understood by a person of ordinary skill in the art. In any disclosed embodiment, the terms “substantially”, “approximately”, “generally”, and “about” may be substituted with “within [a percentage] of” what is specified, where the percentage includes 0.1, 1, 5, and 10 percent.

The foregoing outlines features of several embodiments so that those skilled in the art may better understand the aspects of the disclosure. Those skilled in the art should appreciate that they may readily use the disclosure as a basis for designing or modifying other processes and structures for carrying out the same purposes and/or achieving the same advantages of the embodiments introduced herein. Those skilled in the art should also realize that such equivalent constructions do not depart from the spirit and scope of the disclosure, and that they may make various changes, substitutions, and alterations herein without departing from the spirit and scope of the disclosure. The scope of the invention should be determined only by the language of the claims that follow. The term “comprising” within the claims is intended to mean “including at least” such that the recited listing of elements in a claim are an open group. The terms “a”, “an”, and other singular terms are intended to include the plural forms thereof unless specifically excluded.

1. A composition for prevention or treatment of cancer, the composition comprising:
an arginine mimic (TA-R) selected from the group consisting of trans-4-guanidinomethylcyclohexanecarboxylic acid (TA-R1), amino((((1r,4r)-4-(methoxycarbonyl)cyclohexyl)methyl)amino)methaniminium chloride (TA-R1-OMe), amino(((1r,4r)-4-carboxycyclohexyl)amino)methaniminium chloride (TA-R2), cis-amino(((3-carboxycyclohexyl)methyl)amino)methaniminium chloride (TA-R3), cis-amino(((3 (methoxycarbonyl)cyclohexyl)methyl)amino)methaniminium chloride (TA-R3-OMe), and cis-3-(guanidinomethyl)cyclopentane-1-carboxylic acid (TA-R4), compounds that have a cycloalkane having two or more functional groups off the cycloalkane, derivates thereof, salts thereof, and combinations thereof.

2. The composition of claim 1, wherein the arginine mimic has a concentration in a range of 0.5 to 20% (w/v).

3. The composition of claim 1, wherein the arginine mimic preserves a spatial relationship of a carboxyl group and a guanidinium group of an amino acid.

4. The composition of claim 1, wherein the arginine mimic inhibits viability of cells.

5. The composition of claim 4, wherein the cells comprise cancer cells.

6. The composition of claim 5, wherein the cancer cells are breast cancer cells.

7. The composition of claim 1, wherein the arginine mimic decreases expression of the MYC oncogene.

8. The composition of claim 1, wherein the arginine mimic reduces histone acetylation.

9. The composition of claim 1, wherein the arginine mimic inhibits p300 acetyltransferase activity.

10. The composition of claim 1, wherein the arginine mimic decreases programmed death-ligand 1 (PD-L1) expression in cancer cells thereby blocking T-cell-based immunity.

11-20. (canceled)

21. A composition for prevention or treatment of cancer, prevention, treatment or inhibition of viral or microbial infection or replication, or prevention, treatment or inhibition of a disease, the composition comprising at least one of an arginine mimic (TA-R) or a histidine mimic (TA-H), wherein the at least one of TA-R or TA-H comprises a TA-R selected from the group consisting of trans-4-guanidinomethylcyclohexanecarboxylic acid (TA-R1), amino((((1r,4r)-4-(methoxycarbonyl)cyclohexyl)methyl)amino)methaniminium chloride (TA-R1-OMe), amino(((1r,4r)-4-carboxycyclohexyl)amino)methaniminium chloride (TA-R2), cis-amino(((3-carboxycyclohexyl)methyl)amino)methaniminium chloride (TA-R3), cis-amino(((3 (methoxycarbonyl)cyclohexyl)methyl)amino)methaniminium chloride (TA-R3-OMe), and cis-3-(guanidinomethyl)cyclopentane-1-carboxylic acid (TA-R4), compounds that have a cycloalkane having two or more functional groups off the cycloalkane, derivates thereof, salts thereof, and combinations thereof.

22. (canceled)

23. The composition of claim 21, wherein the at least one of TA-R or TA-H has a concentration in a range of 0.5 to 20% (w/v).

24. The composition of claim 21, wherein the at least one of TA-R or TA-H preserves a spatial relationship of a carboxyl group and a guanidinium group of an amino acid.

25. The composition of claim 21, wherein the at least one of TA-R or TA-H treats or prevents the development or growth of cancer, a viral or microbial infection, or development of another disease.

26. The composition of claim 25, wherein the cancer is skin cancer.

27. The composition of claim 25, wherein the cancer is breast cancer.

28. The composition of claim 21, wherein the at least one of TA-R or TA-H decreases expression of the MYC oncogene.

29. The composition of claim 21, wherein the at least one of TA-R or TA-H reduces histone acetylation.

30. The composition of claim 21, wherein the at least one of TA-R or TA-H inhibits p300 acetyltransferase activity.

31. The composition of claim 21, wherein the at least one of TA-R or TA-H decreases programmed death-ligand 1 (PD-L1) expression in cancer cells thereby blocking T-cell-based immunity.

32-42. (canceled)
